(19) United States
(12) Patent Application Publication
(10) Pub. No.: US 2013/0288233 A1
(43) Pub. Date: Oct. 31, 2013

(54) Selective Reaction Monitoring (SRM) Derived Protein Profiles for Cancer and other Pathologic Entities

(71) Applicant: Rachael Murray, Goodna (AU)

(72) Inventor: Rachael Murray, Goodna (AU)

(73) Assignee: MAP DIAGNOSTICS PTY LTD., Goodna (AU)

(21) Appl. No.: 13/997,352

(22) PCT Filed: Sep. 21, 2011
(86) PCT No.: PCT/AU2011/001218

§ 371 Date: Jun. 24, 2013

(30) Foreign Application Priority Data
Dec. 24, 2010 (AU) 2010905650

(51) Int. Cl. G01N 33/68

(52) U.S. Cl. CPC G01N 33/6893
USPC 435/5; 435/23; 435/15; 435/19

(57) ABSTRACT

The invention relates to a method of detecting and quantifying small peptides derived from proteins from a range of different clinical samples using the Selective Reaction Monitoring (SRM) profiling technique. By targeting these unique peptides which specifically identify particular proteins, the present invention enables multiple samples to be run in a multiplexed fashion in order to identify, diagnose, quantitate and profile a full range of benign and pathologic entities, including but not limited to, the complete range of cancers and the spectrum of inflammatory diseases, including inflammatory cell typing and bone marrow cell typing. The SRM assay is capable of performing clinical blood typing and it can also act as a diagnostic test to identify women at highest risk for cervical cancer base on Human Papillomavirus (HPV) testing.

### FIELD OF THE INVENTION

The invention relates to a method of detecting a platform of small peptides from numerous proteins that enables the profiling of different cancer and other pathological entities using the selective reaction monitoring (SRM) profiling technique, also known as multiple reaction monitoring (MRM).

### BACKGROUND OF THE INVENTION

Currently a wide range of antibody-based detection methods are routinely used to detect a large number of antigens when studying the molecular phenotype of various pathological entities. In fact particular sets of antibodies can be used to separate and identify the wide variety of cancers, including, adenocarcinomas, squamous cell carcinomas, melanomas and mesotheliomas. There are other cohorts of antibodies that are applied to tumours from different body sites to provide not only evidence of the tumours site of origin, but also to provide prognostic and therapeutic guidance. The affinity of any particular antibody is a reflection of the quality of fit between a single antigen binding site and its antigen and is independent of the number of antigenic sites. For this reason it is impossible to quantify the amount of antigen present in a cancer tissue section sample based on antibody binding.

There have been recent technological advances in the field of mass spectrometry, namely the introduction of triple quadrupole instruments which markedly extend the instruments' range of mass detection and to enable sequence analysis using tandem mass spectrometry. This has enabled the application of the mass spectrometry technique selective reaction monitoring (SRM) to be applied as a quantitative tool for protein and peptide analysis to become a reality. The selective reaction monitoring technique works by choosing a number of unique identifying peptides from individual proteins of interest. The targeted peptides of interest are detected in clinical samples using a HPLC tandem Mass spectrometry method.

Breast cancer is the most commonly diagnosed malignancy in Western women and results in death in many cases. Traditionally, cytokeratins have been used as markers to differentiate the basal and luminal types of breast cells and also to define subsets of breast tumours, including basal breast cancer.

Immunohistochemistry based studies (Wetzels et al; Am J Pathol 1991 138: 751-763) demonstrate that cytokeratins 7, 8, 18 and 19 are expressed in luminal breast cells, while cytokeratins 5, 14 and 17 are expressed in the basal/myoepithelial cells. Clinically, differential cytokeratin expression is analysed, however, there are limitations in the specificity and sensitivity of the current methods including the anti-body based detection methods. For example, cytokeratins 5 and 6 are highly homologous proteins and it is difficult to accurately distinguish between these proteins using available antibodies. Furthermore, cytokeratin 5 and the cytokeratin 6 isoforms, A, B, C, D, E and F have been identified (Takahashi et al; The Journal of Biological Chemistry 1995, Vol 270, No 31, 18581-18592).

It is an object of the present invention to provide a technique to accurately distinguish between and quantify homologous proteins such as cytokeratin 5 and the cytokeratin 6 isoforms, A, B, C, D, E and F. Not only can the method according to the present invention separate the cytokeratin 5 and 6 isoforms, but it also capable of concurrently profiling the range of cytokeratins listed below.

### SUMMARY OF THE INVENTION

The present invention provides a method of detecting protein biomarkers using a selective reaction monitoring (SRM) technique wherein the biomarkers are selected from a group consisting of Human proteins:

Pro-opiomelanocortin (and its derivatives, including, Adrenocorticotropic hormone, Melanocyte-stimulating hormone, Beta-endorphin and Met-enkephalin), Alpha-fetoprotein, Serine/threonine-protein kinase receptor R3, Alpha-methylacyl-CoA racemase (aka AMACR), Serum amyloid P-component, Beta-catenin, Apoptosis regulator Bcl-2, B-cell lymphoma 6 protein, Epithelial Cell Adhesion Molecule (aka Ep-CAM), POU domain class 2-associating factor 1, Complement C4-A, Calcitonin, Caldesmon, Calretinin, Neprilysin, Mast/stem cell growth factor receptor (2 isoforms), Integrin alpha-X, Syndecan-1, Alpha-(1,3)-fucosyltransferase, Signal transducer CD24, CD44 antigen, Trans-acting T-cell-specific transcription factor GATA-3, T-cell surface glycoprotein CD1a, B-lymphocyte antigen CD20, Complement receptor type 2, B-cell receptor CD22, Low affinity immunoglobulin epsilon Fc receptor, Glycophorin-A, Interleukin-2 receptor subunit alpha, T-cell surface glycoprotein CD3 (E D G and Z), Tumor necrosis factor receptor superfamily member 8, Platelet endothelial cell adhesion molecule, Myeloid cell surface antigen CD33, Hematopoietic progenitor cell antigen CD34, ADP-ribosyl cyclase 1, T-cell surface glycoprotein CD4, Leukosialin, Receptor-type tyrosine-protein phosphatase C (LCA), Receptor-type tyrosine-protein phosphatase C (LCA)low molecular weight isoform of (LCA) isoform 2, T-cell surface glycoprotein CD5, Neural cell adhesion molecule 1, Carbohydrate sulfotransferase 10, Integrin beta-3, Macrosialin, T-cell antigen CD7, B-cell antigen receptor complex-associated protein alpha chain, T-cell surface glycoprotein CD8 alpha chain, CD99 antigen, Homeobox protein CDX-2, Carcinoembryonic antigen-related cell adhesion molecule 5, Chromogranin-A, Cytokeratin 4, Cytokeratin 5, Cytokeratin 6A, Cytokeratin 6B, Cytokeratin 6C, Cytokeratin 6D, Cytokeratin 6E, Cytokeratin 6F, Cytokeratin 7, Cytokeratin 8, Cytokeratin 14, Cytokeratin 17, Cytokeratin 18, Cytokeratin 19, Cytokeratin 20, Collagen alpha-4(IV) chain, 01/S-specific cyclin-D1, Podoplanin, Desmin, Anoctamin-1, Cadherin-1 (aka E-cadherin), Mucin-1 (aka EMA), Mucin-2, Mucin-5AC, Mucin-6, Coagulation factor VIII, Coagulation factor XIII A chain, Glycoprotein hormones alpha chain, Follitropin subunit beta, Prolactin-inducible protein, Glial fibrillary acidic protein, Somatotropin (Growth Hormone), Solute carrier family 2, facilitated glucose transporter member 1, Glypican-3, Granzyme B, Choriogonadotropin subunit beta, Epidermal growth factor receptor, Receptor tyrosine-protein kinase erbB-2, Receptor tyrosine-protein kinase erbB-3, Receptor tyrosine-protein kinase erbB-4, Melanocyte protein PMEL (aka gp100), Chorionic somatomammotropin hormone, Inhibin alpha chain, Inhibin beta A chain, Inhibin beta B, Inhibin betaC, Inhibin betaE, Antigen KI-67, Lutropin subunit beta, Glycoprotein hormones alpha chain, E3 ubiquitin-protein ligase Mdm2, Melanoma antigen recognized by T-cells 1, DNA mismatch repair protein MIh1, Aortic smooth muscle Actin, DNA mismatch repair protein Msh2, DNA mismatch repair protein Msh6, Myeloperoxidase, Myogenin, Neurofilament light polypeptide, Neurofilament heavy polypeptide, Gamma-enolase, POU domain class 2 transcription factor 2, oestrogen receptor alpha, oestrogen receptor beta, ovamacroglobulin, Cyclin-dependent kinase inhibitor 2A(isoforms 1,2,3), Cellular tumor antigen p53, Cyclin-dependent kinase inhibitor 1C, Tumor protein 63, Catenin delta-1, Prostatic acid phosphatase, Paired box protein Pax-5, Ubiquitin carboxyl-terminal hydrolase isozyme L1, Peptidyl-prolyl cis-trans isomerase NIMA-interacting 4 (aka PIN4), Alkaline phosphatase placental type, Mismatch repair endonuclease PMS2, Progesterone receptor, Prolactin, Prostate-specific antigen (Kallikrein-3), Kallikrein-4, Kallikrein-5, Kallikrein-7, Androgen Receptor, Protein S100-A1, Protein S100-B, Protein S100-A6, Myosin-11 Smooth muscle myosin heavy chain isoform SM1, Synaptophysin, DNA nucleotidylexotransferase, Thyroglobulin, Thyrotropin subunit beta, Homeobox protein Nkx-2.1, Villin-1, Wilms tumor protein, Retinoblastoma-associated protein, Mesothelin, Ubiquitin carboxyl-terminal hydrolase isozyme L1, Pro-neuregulin-1, GP30, Breast cancer type 1 susceptibility protein, Breast cancer type 2 susceptibility protein, Claudin 1, Claudin 2, Claudin 3, Claudin 4, Claudin 5, Claudin 6 Claudin 7, Claudin 16, Isocitrate dehydrogenase [NADP] cytoplasmic, Isocitrate dehydrogenase [NADP] mitochondrial, Follicle-stimulating hormone receptor, Appetite-regulating hormone (Including Ghrelin and Obestatin), Growth hormone secretagogue receptor type 1 (A&B isoforms), GTPase KRas, GTPase NRas, GTPase HRas, Serine/threonine-protein kinase B-raf, Myc proto-oncogene protein, Ig lambda-1 chain C regions, Ig lambda-2 chain C regions, Ig lambda-3 chain C regions, Ig lambda-6 chain C region, Ig lambda-7 chain C region, Ig kappa chain C region, Ig mu chain C region, Ig gamma-1 chain C region, Ig alpha-1 chain C region, Ig alpha-2 chain C region, Ig delta chain C region, Ig epsilon chain C region, Histo-blood group ABO system transferase, Complement C4-A, Complement C4-B, Aquaporin-1, Aquaporin-3, Complement decay-accelerating factor, Band 3 anion transport protein, Ecto-ADP-ribosyltransferase 4, Duffy antigen/chemokine receptor, Galactoside 2-alpha-L-fucosyltransferase 1, Galactoside 2-alpha-L-fucosyltransferase 2, Galactoside 3(4)-L-fucosyltransferase, CD44 antigen, Semaphorin-7A, Kell blood group glycoprotein, Urea transporter 1, Complement receptor type 1, Membrane transport protein XK, Intercellular adhesion molecule 4, Basal cell adhesion molecule, Glycophorin-A, Glycophorin-B, Glycophorin-C, Basigin, UDP-GalNAc:beta-1,3-N-acetylgalactosaminyltransferase 1, CD151 antigen, Blood group Rh(D) polypeptide, Blood group Rh(CE) polypeptide, Erythroid membrane-associated protein, Glycoprotein Xg, and Acetylcholinesterase.

And the following Human Papillomavirus (HPV) proteins, Protein E6, Protein E7, L1 Proteins for High risk type (HPV's) 16, 18, 31, 33, 35, 39, 45, 51, 52, 56, 58, 59, 66 and 68.

In another aspect, the present invention provides a method of selecting optimal SRM peptides and transitions for the proteins according to claim 1 to improve full clinical capacity comprising:

(i) designing a set of SRM transitions using MRM Pilot (AB SCIEX) for each protein biomarker;

(ii) manually evaluating the peptide transitions to ascertain if the protein of interest belongs to a family of homologous proteins, or if the protein has multiple alternative isoforms, or if there are natural variants of these proteins, or if there are known post-translational modifications which have therapeutic significance for the patients;

(iii) If any of these conditions in (ii) are met, then performing in silico digestions to highlight peptides that are capable of identifying these isoforms or modified peptides of interest; and (iv) manually verifying these peptides using NCB! Blast to determine that they were unique peptides for the individual proteins.

Preferably, the method comprises the steps of:

(a) preparing the clinical samples to enable them to be successfully digested;

(b) reducing and alkylating the protein samples; and

(c) digesting the resultant sample with trypsin to provide tryptic peptides.

Preferably, the tryptic peptides are separated using an Ultimate 3000 HPLC with Nanospray® Ion Source and an Acclaim® Pepmap column (Dionex); an QTRAP® 5500 LC/MS/MS (AB SCIEX) system to provide mass spectra; and Multiquant™ software (AB SCIEX) applied to analyse the resultant spectra and multiple transitions for each peptide.

In another aspect, the invention provides a method of detecting the relative or absolute amount of an individual protein isoform, according to the present invention, from each clinical sample processed by the SRM assay.

In another aspect, the method can distinguish between cytokeratin 5 and 6 isoforms using the selective reaction monitoring (SRM) profiling technique.

Preferably, the cytokeratins are used as markers to differentiate between different types of cancer.

In another aspect, the invention provides a method using combinations of the proteins according to claim 1 in SRM based assays to provide a multiplexed diagnostic platform, which would be of use in diagnosing a range of benign and pathologic entities, providing a quantifiable profile for the complete range of cancers, including but not limited to, adenocarcinoma, squamous cell carcinoma, melanoma, mesothelioma, neuroendocrine tumours, lymphoma, and leukaemia, together with identifying proteins from tumours of different organ sites of origin, eg, breast, lung or prostate.

Preferably, the SRM assay is also capable of diagnosing a range of inflammatory diseases, including inflammatory cell typing and bone marrow cell typing.

Preferably, to perform the aforementioned assays, different groups of available SRM's will include but not be limited to the following proteins:

Pro-opiomelanocortin (and its derivatives, including, Adrenocorticotropic hormone, Melanocyte-stimulating hormone, Beta-endorphin and Met-enkephalin), Alpha-fetoprotein, Serine/threonine-protein kinase receptor R3, Alpha-methylacyl-CoA racemase (aka AMACR), Serum amyloid P-component, Beta-catenin, Apoptosis regulator BcI-2, B-cell lymphoma 6 protein, Epithelial Cell Adhesion Molecule (aka Ep-CAM), POU domain class 2-associating factor 1, Complement C4-A, Calcitonin, Caldesmon, Calretinin, Neprilysin, Mast/stem cell growth factor receptor (2 isoforms), Integrin alpha-X, Syndecan-1, Alpha-(1,3)-fucosyltransferase, Signal transducer CD24, CD44 antigen, Trans-acting T-cell-specific transcription factor GATA-3, T-cell surface glycoprotein CD1a, B-lymphocyte antigen CD20, Complement receptor type 2, B-cell receptor CD22, Low affinity immunoglobulin epsilon Fc receptor, Glycophorin-A, Interleukin-2 receptor subunit alpha, T-cell surface glycoprotein CD3 (E D G and Z), Tumor necrosis factor receptor superfamily member 8, Platelet endothelial cell adhesion molecule, Myeloid cell surface antigen CD33, Hematopoietic progenitor cell antigen CD34, ADP-ribosyl cyclase 1, T-cell surface glycoprotein CD4, Leukosialin, Receptor-type tyrosine-protein phosphatase C (LCA), Receptor-type tyrosine-protein phosphatase C (LCA)low molecular weight isoform of (LCA) isoform 2, T-cell surface glycoprotein CD5, Neural cell adhesion molecule 1, Carbohydrate sulfotransferase 10, Integrin beta-3, Macrosialin, T-cell antigen CD7, B-cell antigen receptor complex-associated protein alpha chain, T-cell surface glycoprotein CD8 alpha chain, CD99 antigen, Homeobox protein CDX-2, Carcinoembryonic antigen-related cell adhesion molecule 5, Chromogranin-A, Cytokeratin 4, Cytokeratin 5, Cytokeratin 6A, Cytokeratin 6B, Cytokeratin 6C, Cytokeratin 6D, Cytokeratin 6E, Cytokeratin 6F, Cytokeratin 7, Cytokeratin 8, Cytokeratin 14, Cytokeratin 17, Cytokeratin 18, Cytokeratin 19, Cytokeratin 20, Collagen alpha-4(IV) chain, G1/S-specific cyclin-D1, Podoplanin, Desmin, Anoctamin-1, Cadherin-1 (aka E-cadherin), Mucin-1 (aka EMA), Mucin-2, Mucin-5AC, Mucin-6, Coagulation factor VIII, Coagulation factor XIII A chain, Glycoprotein hormones alpha chain, Follitropin subunit beta, Prolactin-inducible protein, Glial fibrillary acidic protein, Somatotropin (Growth Hormone), Solute carrier family 2, facilitated glucose transporter member 1, Glypican-3, Granzyme B, Choriogonadotropin subunit beta, Epidermal growth factor receptor, Receptor tyrosine-protein kinase erbB-2, Receptor tyrosine-protein kinase erbB-3, Receptor tyrosine-protein kinase erbB-4, Melanocyte protein PMEL (aka gp100), Chorionic somatomammotropin hormone, Inhibin alpha chain, Inhibin beta A chain, Inhibin beta B, Inhibin betaC, Inhibin betaE, Antigen KI-67, Lutropin subunit beta, Glycoprotein hormones alpha chain, E3 ubiquitin-protein ligase Mdm2, Melanoma antigen recognized by T-cells 1, DNA mismatch repair protein Mih1, Aortic smooth muscle Actin, DNA mismatch repair protein Msh2, DNA mismatch repair protein Msh6, Myeloperoxidase, Myogenin, Neurofilament light polypeptide, Neurofilament heavy polypeptide, Gamma-enolase, POU domain class 2 transcription factor 2, oestrogen receptor alpha, oestrogen receptor beta, ovamacroglobulin, Cyclin-dependent kinase inhibitor 2A(isoforms 1,2,3), Cellular tumor antigen p53, Cyclin-dependent kinase inhibitor 1C, Tumor protein 63, Catenin delta-1, Prostatic acid phosphatase, Paired box protein Pax-5, Ubiquitin carboxyl-terminal hydrolase isozyme L1, Peptidyl-prolyl cis-trans isomerase NIMA-interacting 4 (aka PIN4), Alkaline phosphatase placental type, Mismatch repair endonuclease PMS2, Progesterone receptor, Prolactin, Prostate-specific antigen (Kallikrein-3), Kallikrein-4, Kallikrein-5, Kallikrein-7, Androgen Receptor, Protein S100-A1, Protein S100-B, Protein S100-A6, Myosin-11 Smooth muscle myosin heavy chain isoform SM1, Synaptophysin, DNA nucleotidylexotransferase, Thyroglobulin, Thyrotropin subunit beta, Homeobox protein Nkx-2.1, Villin-1, Wilms tumor protein, Retinoblastoma-associated protein, Mesothelin, Ubiquitin carboxyl-terminal hydrolase isozyme L1, Pro-neuregulin-1, GP30, Breast cancer type 1 susceptibility protein, Breast cancer type 2 susceptibility protein, Claudin 1, Claudin 2, Claudin 3, Claudin 4, Claudin 5, Claudin 6 Claudin 7, Claudin 16, Isocitrate dehydrogenase [NADP] cytoplasmic, Isocitrate dehydrogenase [NADP] mitochondrial, Follicle-stimulating hormone receptor, Appetite-regulating hormone (Including Ghrelin and Obestatin), Growth hormone secretagogue receptor type 1 (A&B isoforms), GTPase KRas, GTPase NRas, GTPase HRas, Serine/threonine-protein kinase B-raf, Myc proto-oncogene protein, Ig lambda-1 chain C regions, Ig lambda-2 chain C regions, Ig lambda-3 chain C regions, Ig lambda-6 chain C region, Ig lambda-7 chain C region, Ig kappa chain C region, Ig mu chain C region, Ig gamma-1 chain C region, Ig alpha-1 chain C region, Ig alpha-2 chain C region, Ig delta chain C region, Ig epsilon chain C region.

In another aspect, the invention provides a method of to provide a diagnostic test to identify women at highest risk for cervical cancer using combinations of the following Human Papillomavirus (HPV) proteins in an SRM based assay:

Protein E6, Protein E7, L1 Proteins for High risk type HPV's 16, 18, 31, 33, 35, 39, 45, 51, 52, 56, 58, 59, 66 and 68.

In another aspect, the invention provides a method of using combinations of the following proteins to provide an SRM based multiplexed diagnostic platform for use in detecting and quantifying the range of proteins that form the basis of clinical blood typing:

Histo-blood group ABO system transferase, Complement C4-A, Complement C4-B, Aquaporin-1, Aquaporin-3, Complement decay-accelerating factor, Band 3 anion transport protein, Ecto-ADP-ribosyltransferase 4, Duffy antigen/chemokine receptor, Galactoside 2-alpha-L-fucosyltransferase 1, Galactoside 2-alpha-L-fucosyltransferase 2, Galactoside 3(4)-L-fucosyltransferase, CD44 antigen, Semaphorin-7A, Kell blood group glycoprotein, Urea transporter 1, Complement receptor type 1, Membrane transport protein XK, Intercellular adhesion molecule 4, Basal cell adhesion molecule, Glycophorin-A, Glycophorin-B, Glycophorin-C, Basigin, UDP-GaI:beta-1,3-N-acetylgalactosaminyltransferase 1, CD151 antigen, Blood group Rh(D) polypeptide, Blood group Rh(CE) polypeptide, Erythroid membrane-associated protein, Glycoprotein Xg, Acetylcholinesterase.

In another aspect, the invention provides a method for an SRM based assay to quantifiably separate the 4 isoforms of EGFR protein comprising the steps of:

(i) separating the 4 isoforms of the protein by a more sensitive and accurate SRM assay than the currently used antibody based detection methods;

(ii) targeting specific peptides, to identify and quantify the many natural variants of these proteins that are caused by mutation and have been detected in lung, colorectal and breast cancers; and

(iii) detecting peptides of interest from the various isoforms which have been modified by post-translational modifications such as, but not limited to phosphorylation, glycosylation and ubiquitination.

In another aspect, the invention provides a method for an SRM based assay to quantifiably separate the 4 isoforms of Receptor tyrosine-protein kinase erbB protein comprising the steps of:

(i) separating the 4 isoforms of the protein by SRM, an assay that is more sensitive and accurate than the currently used antibody-based detection methods;

(ii) targeting specific peptides, to identify and quantify the many natural variants of Receptor tyrosine-protein kinase erbB-2 that are caused by in frame mutations and have been implicated in lung adenocarcinoma, gastric adenocarcinoma, ovarian cancer and glioma;

(iii) detecting peptides of interest from the various isoforms which have been modified by post-translational modifications such as, but not limited to phosphorylation and glycosylation.

Preferably, the cancer includes the basal and luminal types of breast cancer cells.

In another aspect, the invention provides a method for mass spectrometry analysis of a sample comprising cytokeratins 5 and 6 using SRM.

In another aspect, the invention provides a kit for use in mass spectrometry analysis of a sample comprising cytokeratins 5 and 6 and reagents to enable the analysis.

In another aspect, the invention provides a method to distinguish between small chain peptides using the SRM technique.

Preferably, the peptides are cytokeratins.

Preferably, the cytokeratins are CK5 or CK6.

In another aspect, the invention provides a method of detecting small chain peptides using SRM technique wherein the peptides are used as markers to detect different types of cancer.

Preferably, the cancer includes breast cancer and the SRM technique can separate not only the basal and luminal types of breast cancer cells, but also all of the molecular based subtypes of breast cancer.

In another aspect, the invention provides a method according to any one of the preceding claims to study a range of cell lines, benign and tumour cell lysates derived either from formalin-fixed cells or tissues embedded in paraffin blocks, fresh or fresh frozen tissue, biological body fluids including but not limited to blood, serum, urine, cerebrospinal fluid, pleural fluid, peritoneal fluid, bone marrow, nipple aspirate fluid, samples from a cytology thin layer vial containing either SurePath™ preservative fluid or PreservCyt™ solution, and fine needle aspirate (FNA) samples.

In another aspect, the invention provides a method for evaluating the prognosis or therapeutic implications for a patient, said method comprising detecting expression of at least one biomarker in a sample from said patient using the SRM technique, wherein said biomarker is selected from a group consisting of the biomarkers according to the present invention.

Preferably, the biomarkers are selected from the group consisting of Cytokeratin 4, Cytokeratin 5, Cytokeratin 6A, Cytokeratin 6B, Cytokeratin 6C, Cytokeratin 6D, Cytokeratin 6E, Cytokeratin 6F, Cytokeratin 7, Cytokeratin 8, Cytokeratin 14, Cytokeratin 17, Cytokeratin 18, Cytokeratin 19 and Cytokeratin 20.

In another aspect, the invention provides a mass spectrometry based kit to perform analysis of a sample including protein profiling.

Preferably, the kit may be in the form of a database interface which aligns information generated by a mass spectrometer to produce quantifiable parameter based reports for clients.

Preferably, the kit comprises cytokeratins 5 and 6 and isoforms thereof and reagents or software to enable the analysis.

### DETAILED DESCRIPTION OF THE INVENTION

The invention is a multiplexed diagnostic assay that will serve as a routine test in cancer and disease diagnostics in the pathology or clinical research industries. This multiplexed assay is based on the method of detecting small peptides from proteins using the Mass Spectrometry based Selective Reaction Monitoring (SRM) technique, also known as Multiple Reaction Monitoring (MRM). The inventive aspect of this assay is the application of this SRM technology to the full range of protein biomarkers, approximately 200 proteins, which the pathology industry currently tests for on a daily basis in their Anatomical pathology, Immunology and Haematology departments.

Currently, the pathology industry uses a variety of antibody-based tests to detect these protein biomarkers. These antibody-based tests have a number of limitations, as they are not quantitative in immunohistochemistry applications, the antibody reactions are notoriously non-specific and only a very limited number of proteins can be detected in a single sample.

By using SRM to target unique peptides which specifically identify a particular protein, the present invention enables these assays to be run in a multiplexed fashion in order allow pathologists to identify, diagnose, quantitate and profile a full range of benign and pathologic entities, including but not limited to, the complete range of cancers, including, adenocarcinoma, squamous cell carcinoma, melanoma, mesothelioma, neuroendocrine tumours, lymphoma, and leukaemia and also the spectrum of inflammatory diseases, including inflammatory cell typing and bone marrow cell typing. The assay can also provide pathologists with prognostic and therapeutic guidance. The SRM assay is capable of performing clinical blood typing and it can also act as a diagnostic test to identify women at highest risk for cervical cancer based on Human Papillomavirus (HPV) testing.

The multiplexed SRM assays have been specifically designed to detect these human protein biomarkers and Human Papillomavirus proteins according to the present invention.

The SRM assays can be designed to detect a wider range of proteins in the future. The SRM platform according to the present invention has been designed with a view to being able to provide laboratories with quantitative diagnostic profiles for a range of different benign and pathological entities based on the protein expression profiles of up to hundreds of different proteins.

The assays are easily multiplexed, so that a large number of markers can be tested on a single sample. Currently, this technology is capable of detecting and providing absolute quantitation for up to 350 proteins in a single drop of blood in a time frame of 35 minutes. The SRM technology when applied to clinical samples is fully compatible with the current diagnostic processes and timeframes.

In one embodiment, the present invention permits application of this selective reaction monitoring technique as a multiplexed protein profiling platform to be used to profile and study a wide variety of cancers. The technique can be applied to study a range of cell lines, benign and cancerous cell lysates, derived either from formalin-fixed cells or tissues embedded in paraffin blocks, fresh or fresh frozen tissue, biological body fluids including but not limited to blood, serum, urine, cerebrospinal fluid, pleural fluid, peritoneal fluid, bone marrow, nipple aspirate fluid, samples from a cytology thin layer vial containing either SurePath™ preservative fluid or PreservCyt™ solution and fine needle aspirate (FNA) samples.

Breast cancer is just one type of cancer that has been analysed using this multiple reaction monitoring methodology.

In a preferred embodiment, this MRM technology has been applied to study breast cancer cell lines. Results based on this study demonstrate the capability of the SRM technology to distinguish between a homologous group of proteins, namely cytokeratin 5 and the various cytokeratin 6 isoforms. Traditionally, cytokeratins 5 and 6 have been used as markers to diagnose basal breast cancer. The available antibodies for cytokeratin 5 and the cytokeratin 6 isoforms are limited as they are not able to distinguish between these highly homologous proteins. According to the present invention, the method relates to the selective reaction monitoring (SRM) which is a highly specific and sensitive mass spectrometry (MS) technique that can selectively quantify multiple proteins within complex mixtures. Hence, the present invention applies a targeted MS approach using SRM to identify and characterize cytokeratin expression in a number of breast cancer cell lines. The present invention is capable of separating the highly homologous cytokeratin 5 and the cytokeratin 6 isoforms.

The group of epithelial keratins (K) also demonstrate specific expression patterns in a range of human tumours. Several of them (particularly K5, K7, K8/K18, K19 and K20) have great importance in immunohistochemical diagnosis of carcinomas, especially in precise classification and subtyping. Hence the present invention can be applied to the range of human tumours as a specific and quantitative multiplexed diagnostic assay.

### Technical Methodology

### Standard Operating Procedure

### Example 1

For each protein biomarker a set of SRM transitions was designed using MRM Pilot (AB SCIEX). Peptide transitions were then manually evaluated to ascertain if the protein of interest belongs to a family of homologous proteins, or if the protein has multiple alternative isoforms, or if there are natural variants of these proteins, or if there are known post-translational modifications which have therapeutic significance for the patients. If any of these conditions held true, then in silico digestions were performed to highlight peptides that were capable of identifying these isoforms or modified peptides of interest. These peptides were also manually verified using NCBI Blast to determine if they were unique peptides for the individual proteins. Processing of the samples was performed by precipitating the cellular proteins, reducing and alkylating the sample and then digesting the resultant sample with trypsin. Other enzymes may be added to digest the sample proteins if required. The tryptic peptides were then separated using an Ultimate 3000 HPLC with Nanospray® Ion Source and an Acclaim® Pepmap column (Dionex). The mass spectrometry analysis was performed on a QTRAP® 5500 LC/MS/MS (AB SCIEX) system. Then Multiquant™ software (AB SCIEX) was used to analyse the resultant spectra and multiple transitions for each peptide.

### Example 2

Modification and variation on Example 1 include a range of quantitative methods using either mTRAQ® reagents (AB SCIEX), or heavy peptides of AQUA type, or even label-free quantification combined with selected reaction monitoring, to serve as an assay standard, have all been used to provide relative and absolute quantification of the protein biomarkers of interest in complex biological samples.

Furthermore, it may be possible to incorporate Imaging Mass Spectrometry into the clinical analysis of these proteins, although the technology is currently not sufficiently advanced to allow this technique to be routinely incorporated into clinical practice in the pathology testing.

On the other hand, SWATH™ Acquisition technology (AB SCIEX) may come to play a more pivotal role in the quantitation of various peptides due to the different database searching system utilized in this methodology. Preliminary studies utilizing this technology are demonstrating quantitative performance comparable to leading triple quadrupole instruments, however further evaluation will be needed prior to incorporating this methodology in an appropriate manner.

### Technical Sample Preparation Method

Preferably, the method according to the present invention comprises the steps of:

(a) preparing the clinical samples to enable them to be successfully digested, eg Paraffin embedded tissue needs to be dewaxed, and placed in an appropriate buffer to allow enzymatic digestion to be performed; depleting high abundance proteins from blood or serum samples may be performed; immunopurification comprising capture and extraction of protein of interest in said sample with appropriate antibodies may be used, and various enrichment protocols may be used to increase the concentration of specific groups of peptides in the sample.

(b) reducing and alkylating the protein samples; and

(c) digesting the resultant sample with an appropriate enzyme, such as trypsin. Other methods of proteolysis exist, however enzymatic digestion is specific, and in silico digestion has been performed to determine which specific enzyme can produce the best proteolytic peptides, which are capable of characterising a particular diagnostic protein isoform, protein variant or post-translational modification. Other enzymes may be added to digest the sample proteins if required.

A method has been developed in which the SRM methodology can be used to separate not only the basal and luminal types of breast cancer cells, but can also separate the molecular subtypes of breast cancer that have previously been separated by their genetic expression profiles. Gene expression analyses have defined six tumour subtypes (luminal A, luminal B, HER2-enriched, normal-like, basal-like and claudin-low). These subtypes are predictive of relapse-free and overall survival times, and are also predictive of responsiveness to chemotherapy.

A method has been developed for an SRM based assay to quantifiably separate the 4 isoforms of EGFR protein. This SRM method of separating the 4 isoforms of the protein is a more sensitive and accurate assay than the currently used antibody based detection methods. In this SRM assay several different enzymes (Ie trypsin, chymotrypsin and pepsin) are used to digest the tissue, however alternative enzymes could be used. This particular SRM method can also target specific peptides, to identify and quantify the many natural variants of these proteins that are caused by the various mutations and have been detected in lung, colorectal and breast cancers. This SRM method can also detect peptides of interest from the various isoforms which have been modified by post-translational modifications such as, but not limited to phosphorylation, glycosylation and ubiquitination. This method has been applied to all of the EGFR protein isoforms and modifications described in Uniprot (http://www.uniprot.org/).

A method has been developed for an SRM based assay to quantifiably separate the 4 isoforms of Receptor tyrosine-protein kinase erbB protein. Receptor tyrosine-protein kinase erbB protein overexpression is observed in 25%-30% of primary breast cancers. This SRM method of separating the 4 isoforms of the protein is a more sensitive and accurate assay, than the currently used antibody based detection methods. This SRM method can also target specific peptides, to identify and quantify the many natural variants of Receptor tyrosine-protein kinase erbB-2 that are caused by in frame mutations and have been implicated in lung adenocarcinoma, gastric adenocarcinoma, ovarian cancer and glioma. This SRM method can also detect peptides of interest from the various isoforms which have been modified by post-translational modifications such as, but not limited to phosphorylation and glycosylation. As mentioned in the EGFR protocol above, different enzymes are applied to this SRM assay for Receptor tyrosine-protein kinase erbB-2. This SRM method has been applied to all of the Receptor tyrosine-protein kinase erbB protein isoforms and modifications described in Uniprot.

While considerable emphasis has been placed herein on the specific features of the preferred embodiment, it will be appreciated that many additional features can be added and that many changes can be made in the preferred embodiment without departing from the principles of the invention. These and other changes in the preferred embodiment of the invention will be apparent to those skilled in the art from the disclosure herein, whereby it is to be distinctly understood that the foregoing descriptive matter is to be interpreted merely as illustrative of the invention and not as a limitation.

1. A method of detecting protein biomarkers using a selective reaction monitoring (SRM) technique wherein the biomarkers are human proteins selected from Pro-opiomelanocortin (and its derivatives, including, Adrenocorticotropic hormone, Melanocyte-stimulating hormone, Beta-endorphin and Met-enkephalin), Alpha-fetoprotein, Serine/threonine-protein kinase receptor R3, Alpha-methylacyl-CoA racemase (aka AMACR), Serum amyloid P-component, Beta-catenin, Apoptosis regulator Bcl-2, B-cell lymphoma 6 protein, Epithelial Cell Adhesion Molecule (aka Ep-CAM), POU domain class 2-associating factor 1, Complement C4-A, Calcitonin, Caldesmon, Calretinin, Neprilysin, Mast/stem cell growth factor receptor (2 isoforms), Integrin alpha-X, Syndecan-1, Alpha-(1,3)-fucosyltransferase, Signal transducer CD24, CD44 antigen, Trans-acting T-cell-specific transcription factor GATA-3, T-cell surface glycoprotein CD1a, B-lymphocyte antigen CD20, Complement receptor type 2, B-cell receptor CD22, Low affinity immunoglobulin epsilon Fc receptor, Glycophorin-A, Interleukin-2 receptor subunit alpha, T-cell surface glycoprotein CD3 (E D G and Z), Tumor necrosis factor receptor superfamily member 8, Platelet endothelial cell adhesion molecule, Myeloid cell surface antigen CD33, Hematopoietic progenitor cell antigen CD34, ADP-ribosyl cyclase 1, T-cell surface glycoprotein CD4, Leukosialin, Receptor-type tyrosine-protein phosphatase C (LCA), Receptor-type tyrosine-protein phosphatase C (LCA)low molecular weight isoform of (LCA) Isoform 2, T-cell surface glycoprotein CD5, Neural cell adhesion molecule 1, Carbohydrate sulfotransferase 10, Integrin beta-3, Macrosialin, T-cell antigen CD7, B-cell antigen receptor complex-associated protein alpha chain, T-cell surface glycoprotein CD8 alpha chain, CD99 antigen, Homeobox protein CDX-2, Carcinoembryonic antigen-related cell adhesion molecule 5, Chromogranin-A, Cytokeratin 4, Cytokeratin 5, Cytokeratin 6A, Cytokeratin 6B, Cytokeratin 6C, Cytokeratin 6D, Cytokeratin 6E, Cytokeratin 6F, Cytokeratin 7, Cytokeratin 8, Cytokeratin 14, Cytokeratin 17, Cytokeratin 18, Cytokeratin 19, Cytokeratin 20, Collagen alpha-4(IV) chain, G1/S-specific cyclin-D1, Podoplanin, Desmin, Anoctamin-1, Cadherin-1 (aka E-cadherin), Mucin-1 (aka EMA), Mucin-2, Mucin-5AC, Mucin-6, Coagulation factor VIII, Coagulation factor XIII A chain, Glycoprotein hormones alpha chain, Follitropin subunit beta, Prolactin-inducible protein, Glial fibrillary acidic protein, Somatotropin (Growth Hormone), Solute carrier family 2, facilitated glucose transporter member 1, Glypican-3, Granzyme B, Choriogonadotropin subunit beta, Epidermal growth factor receptor, Receptor tyrosine-protein kinase erbB-2, Receptor tyrosine-protein kinase erbB-3, Receptor tyrosine-protein kinase erbB-4, Melanocyte protein PMEL (aka gp100), Chorionic somatomammotropin hormone, Inhibin alpha chain, Inhibin beta A chain, Inhibin beta B, Inhibin betaC, Inhibin betaE, Antigen KI-67, Lutropin subunit beta, Glycoprotein hormones alpha chain, E3 ubiquitin-protein ligase Mdm2, Melanoma antigen recognized by T-cells 1, DNA mismatch repair protein Mlh1, Aortic smooth muscle Actin, DNA mismatch repair protein Msh2, DNA mismatch repair protein Msh6, Myeloperoxidase, Myogenin, Neurofilament light polypeptide, Neurofilament heavy polypeptide, Gamma-enolase, POU domain class 2 transcription factor 2, oestrogen receptor alpha, oestrogen receptor beta, ovamacroglobulin, Cyclin-dependent kinase inhibitor 2A(isoforms 1,2,3), Cellular tumor antigen p53, Cyclin-dependent kinase inhibitor 1C, Tumor protein 63, Catenin delta-1, Prostatic acid phosphatase, Paired box protein Pax-5, Ubiquitin carboxyl-terminal hydrolase isozyme L1, Peptidyl-prolyl cis-trans isomerase NIMA-interacting 4 (aka PIN4), Alkaline phosphatase placental type, Mismatch repair endonuclease PMS2, Progesterone receptor, Prolactin, Prostate-specific antigen (Kallikrein-3), Kallikrein-4, Kallikrein-5, Kallikrein-7, Androgen Receptor, Protein S100-A1, Protein S100-B, Protein S100-A6, Myosin-11 Smooth muscle myosin heavy chain isoform SM1, Synaptophysin, DNA nucleotidylexotransferase, Thyroglobulin, Thyrotropin subunit beta, Homeobox protein Nkx-2.1, Villin-1, Wilms tumor protein, Retinoblastoma-associated protein, Mesothelin, Ubiquitin carboxyl-terminal hydrolase isozyme L1, Pro-neuregulin-1, GP30, Breast cancer type 1 susceptibility protein, Breast cancer type 2 susceptibility protein, Claudin 1, Claudin 2, Claudin 3, Claudin 4, Claudin 5, Claudin 6 Claudin 7, Claudin 16, Isocitrate dehydrogenase [NADP] cytoplasmic, Isocitrate dehydrogenase [NADP] mitochondrial, Follicle-stimulating hormone receptor, Appetite-regulating hormone (Including Ghrelin and Obestatin), Growth hormone secretagogue receptor type 1 (A&B isoforms), GTPase KRas, GTPase NRas, GTPase HRas, Serine/threonine-protein kinase B-raf, Myc proto-oncogene protein, Ig lambda-1 chain C regions, Ig lambda-2 chain C regions, Ig lambda-3 chain C regions, Ig lambda-6 chain C region, Ig lambda-7 chain C region, Ig kappa chain C region, Ig mu chain C region, Ig gamma-1 chain C region, Ig alpha-1 chain C region, Ig alpha-2 chain C region, Ig delta chain C region, Ig epsilon chain C region, Histo-blood group ABO system transferase, Complement C4-A, Complement C4-B, Aquaporin-1, Aquaporin-3, Complement decay-accelerating factor, Band 3 anion transport protein, Ecto-ADP-ribosyltransferase 4, Duffy antigen/chemokine receptor, Galactoside 2-alpha-L-fucosyltransferase 1, Galactoside 2-alpha-L-fucosyltransferase 2, Galactoside 3(4)-L-fucosyltransferase, CD44 antigen, Semaphorin-7A, Kell blood group glycoprotein, Urea transporter 1, Complement receptor type 1, Membrane transport protein XK, Intercellular adhesion molecule 4, Basal cell adhesion molecule, Glycophorin-A, Glycophorin-B, Glycophorin-C, Basigin, UDP-GalNAc:beta-1,3-N-acetylgalactosaminyltransferase 1, CD151 antigen, Blood group Rh(D) polypeptide, Blood group Rh(CE) polypeptide, Erythroid membrane-associated protein, Glycoprotein Xg, and Acetylcholinesterase, and the Human Papillomavirus (HPV) proteins, Protein E6, Protein E7, L1 Proteins for High risk type (HPV's) 16, 18, 31, 33, 35, 39, 45, 51, 52, 56, 58, 59, 66 and 68.

2. A method of selecting optimal SRM peptides and transitions for the protein biomarkers in the method according to claim 1 to improve full clinical capacity comprising:
(i) designing a set of SRM transitions using MRM Pilot (AB SCIEX) for each protein biomarker;
(ii) manually evaluating the peptide transitions to ascertain if the protein of interest belongs to a family of homologous proteins, or if the protein has multiple alternative isoforms, or if there are natural variants of these proteins, or if there are known post-translational modifications which have therapeutic significance for the patients;
(iii) If any of these conditions in (ii) are met, then performing in silico digestions to highlight peptides that are capable of identifying these isoforms or modified peptides of interest; and
(iv) manually verifying these peptides using NCBI Blast to determine if they were unique peptides for the individual proteins.

3. The method according to claim 1 comprising the steps of:
(a) reducing and alkylating proteins in a clinical sample; and
(b) digesting the resultant proteins with trypsin to provide tryptic peptides.

4. (canceled)

5. A method according to claim 1, comprising detecting the relative or absolute amount of individual isoforms of the protein biomarkers in a clinical sample processed by the SRM assay.

6. A method according to claim 1, comprising distinguishing between cytokeratin 5 and 6 isoforms.

7. The method according to claim 6 wherein the cytokeratins are used as markers to differentiate between different types of cancer.

8. A method according to claim 1, comprising using combinations of the protein biomarkers in SRM based assays to provide a multiplexed diagnostic platform, wherein the platform is used for diagnosis of a range of benign and pathologic entities, providing a quantifiable profile for cancers comprising adenocarcinoma, squamous cell carcinoma, melanoma, mesothelioma, neuroendocrine tumours, lymphoma, and leukaemia and identifying proteins from tumours of different organ sites of origin, comprising breast, lung or prostate.

9. A method according to claim 1, comprising using combinations of the protein biomarkers in SRM based assays to provide a multiplexed diagnostic platform, wherein the platform is used for diagnosis of inflammatory diseases, comprising inflammatory cell typing and bone marrow cell typing.

10. (canceled)

11. A method according to claim 1, wherein the detection of the protein biomarkers is used in a diagnostic test to identify women at highest risk for cervical cancer using combinations of Human Papillomavirus (HPV) proteins Protein E6, Protein E7, L1 Proteins for High risk type HPV's 16, 18, 31, 33, 35, 39, 45, 51, 52, 56, 58, 59, 66 and 68.

12. A method according to claim 1, comprising using combinations of the the protein biomarkers in SRM based assays to provide a multiplexed diagnostic platform, wherein the platform is used for detection and quantitation of proteins that form the basis of clinical blood typing, comprising Histo-blood group ABO system transferase, Complement C4-A, Complement C4-B, Aquaporin-1, Aquaporin-3, Complement decay-accelerating factor, Band 3 anion transport protein, Ecto-ADP-ribosyltransferase 4, Duffy antigen/chemokine receptor, Galactoside 2-alpha-L-fucosyltransferase 1, Galactoside 2-alpha-L-fucosyltransferase 2, Galactoside 3(4)-L-fucosyltransferase, CD44 antigen, Semaphorin-7A, Kell blood group glycoprotein, Urea transporter 1, Complement receptor type 1, Membrane transport protein XK, Intercellular adhesion molecule 4, Basal cell adhesion molecule, Glycophorin-A, Glycophorin-B, Glycophorin-C, Basigin, UDP-GalNAc:beta-1,3-N-acetylgalactosaminyltransferase 1, CD151 antigen, Blood group Rh(D) polypeptide, Blood group Rh(CE) polypeptide, Erythroid membrane-associated protein, Glycoprotein Xg, Acetylcholinesterase.

13. A method according to claim 1, comprising quantifiably separating isoforms of EGFR protein comprising the steps of:
(i) targeting specific peptides, to identify and quantify variants of the isoforms that are caused by mutation and have been detected in lung, colorectal and breast cancers; and
(ii) detecting peptides of interest from the various isoforms which have been modified by post-translational modifications comprising phosphorylation, glycosylation and ubiquitination.

14. A method according to claim 1, comprising quantifiably separating isoforms of Receptor tyrosine-protein kinase erbB protein comprising the steps of:
(ii) targeting specific peptides, to identify and quantify variants of Receptor tyrosine-protein kinase erbB-2 that are caused by in frame mutations and have been implicated in lung adenocarcinoma, gastric adenocarcinoma, ovarian cancer and glioma; and
(iii) detecting peptides of interest from the various isoforms which have been modified by post-translational modifications comprising phosphorylation and glycosylation.

15. The method according to claim 7 wherein the cancer comprises basal and luminal types of breast cancer cells.

16. A method for mass spectrometry analysis of a sample comprising cytokeratins 5 and 6 using SRM.

17. A kit for use in mass spectrometry analysis of a sample comprising cytokeratins 5 and 6 and reagents to enable the analysis.

18. A method according to claim 1, comprising distinguishing between small chain peptides using the SRM technique.

19. The method according to claim 18 wherein the peptides are cytokeratins.

20. The method according to claim 19 wherein the cytokeratins are CK5 or CK6.

21. A method according to claim 18, wherein the peptides are used as markers to detect different types of cancer.

22. The method according to claim 21 wherein the cancer comprises breast cancer and the SRM technique is used to detect basal and luminal types of breast cancer cells and molecular based subtypes of breast cancer.

23. The method according to claim 21 wherein the peptides are cytokeratins.

24-25. (canceled)

26. A method according to claim 1, wherein detection of expression of at least one of the protein biomarkers is used for evaluating the prognostic or therapeutic implications for a patient.

27. The method according to claim 1 wherein the biomarkers are selected from the group consisting of Cytokeratin 4, Cytokeratin 5, Cytokeratin 6A, Cytokeratin 6B, Cytokeratin 6C, Cytokeratin 6D, Cytokeratin 6E, Cytokeratin 6F, Cytokeratin 7, Cytokeratin 8, Cytokeratin 14, Cytokeratin 17, Cytokeratin 18, Cytokeratin 19 and Cytokeratin 20.

28-30. (canceled)
